(19) United States
(12) Patent Application Publication
(10) Pub. No.: US 2003/0098363 A1
(43) Pub. Date: May 29, 2003

(54) Scent dispensing device

(76) Inventor: Dana S. Stanley, Orlando, FL (US)

Correspondence Address: HARSHAW RESEARCH INCORPORATED, P O BOX 418, OTTAWA, KS, 66067, US

(21) Appl. No.: 09/995,394

(22) Filed: Nov. 27, 2001

(51) Int. Cl. A61L 9/04

(52) U.S. Cl. 239/55; 239/57; 239/59

(57) ABSTRACT

A scent dispensing device includes a first sleeve having open and closed ends and defining linearly spaced apart apertures. A second sleeve is slidably received in the first sleeve and defines a slot therealong. The second sleeve includes a handle at one end for rotation of the second sleeve and a plugged end through which a scent soaked absorbent material may be inserted. The second sleeve is rotatable within the first sleeve between one configuration in which the slot and apertures are not aligned and another configuration in which the slot and apertures are aligned whereby to dispense the scent. A hook member is threadably coupled to the second sleeve handle such that the device may be hung from a tree branch. A pair of At opposed arrow tips also enable the device to be inserted into the ground.

### BACKGROUND OF THE INVENTION

The present invention relates generally to scent dispensers and, more particularly, to a scent dispensing device which can be stored in a hunter's quiver, rotatably opened to dispense a selected scent and hung or inserted into the ground during use.

Bow hunting has become a popular mode of hunting, especially hunting for deer. Bow hunters frequently use particular scents to attract deer to the hunter's stationary location which allows the hunter to remain concealed. A disadvantage to a hunter walking around his vicinity spreading scent is that the animals which he is hunting may smell the hunter's own natural scent and be scared away.

Various dispensing devices have been proposed for dispensing scent from a distance, such as with scent dispensing arrows, or with hanging dispensers that require hanging from the end of a branch. U.S. Pat. Nos. 4,881,743 and 5,123,657 are representative of existing proposals. Although assumably effective for their intended purposes, the existing devices do not provide the simplicity of rotational sleeves which can alternatively be inserted into the ground or hung from virtually any appendage of a tree, bush, or the like.

Therefore, it is desirable to have a scent dispensing device that can be carried in a hunter's quiver and then inserted into the ground or hung from a tree, as desired. Further, it is desirable to have a scent dispensing device which dispenses a scent upon rotation of a pair of sleeves to align apertures in one sleeve with a slot in the other. Finally, it is desirable to have a device in which a scent absorbing material may be conveniently removed from the device and replaced.

### SUMMARY OF THE INVENTION

A scent dispensing device according to the present invention includes a first sleeve having a closed first end and an open second end and defining a plurality of apertures longitudinally spaced apart in a linear configuration therebetween. A second sleeve is slidably received within the first sleeve and thus may be slidably inserted or removed via the open second end. The second sleeve includes a handle member with a stopper that prevents the handle member from being inserted into the first sleeve and which enables a user to rotate the second sleeve within the first sleeve. The second sleeve defines at least one elongate slot extending between the handle member and an opposing end. That opposing end includes a plug that may be removed for insertion of an absorbent material into the second sleeve that may be soaked with a selected scent. Therefore, the second sleeve may be selectively rotated such that a slot in the second sleeve is aligned with a linear row of apertures, this configuration allowing the scent odor to be dispensed from the device. The second sleeve may be rotated such that the slot and apertures are not in alignment when the dispensing of scent is no longer desired. The scent dispensing device further includes structures enabling it to be hung from a tree appendage or inserted into the ground.

Therefore, a general object of this invention is to provide a scent dispensing device which includes a perforated outer sleeve and a slotted inner sleeve that cooperate to dispense a scent upon proper rotational alignment.

Another object of this invention is to provide a scent dispensing device, as aforesaid, which includes a scented absorbent material that is easily removable and replaceable.

Still another object of this invention is to provide a scent dispensing device, as aforesaid, which includes an arrow configuration that may be carried in a bow hunter's quiver.

Yet another object of this invention is to provide a scent dispensing device, as aforesaid, which is easy and economical to manufacture.

A further object of this invention is to provide a scent dispensing device, as aforesaid, which may be hung from a tree or inserted into the ground during use.

Other objects and advantages of this invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a scent dispensing device according to a preferred embodiment of the present invention;

FIG. 2 is a fragmentary perspective view of a first sleeve removed from the scent dispensing device as in FIG. 1;

FIG. 3a is a side view of a second sleeve removed from the scent dispensing device go as in FIG. 1;

FIG. 3b is a perspective view of the second sleeve as in FIG. 3a;

FIG. 4a is a perspective view of a hook member removed from the scent dispensing device as in FIG. 1;

FIG. 4b is an end view of the hook member as in FIG. 4a; and

FIG. 5 is a fragmentary perspective view of the scent dispensing device as in FIG. 1 with the second sleeve partially removed therefrom.

### DESCRIPTION OF THE PREFERRED EMBODIMENT

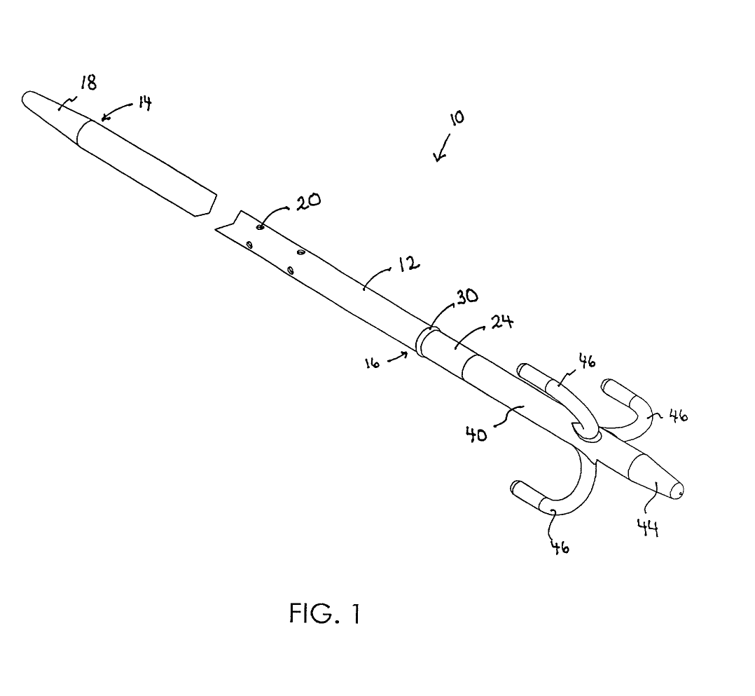

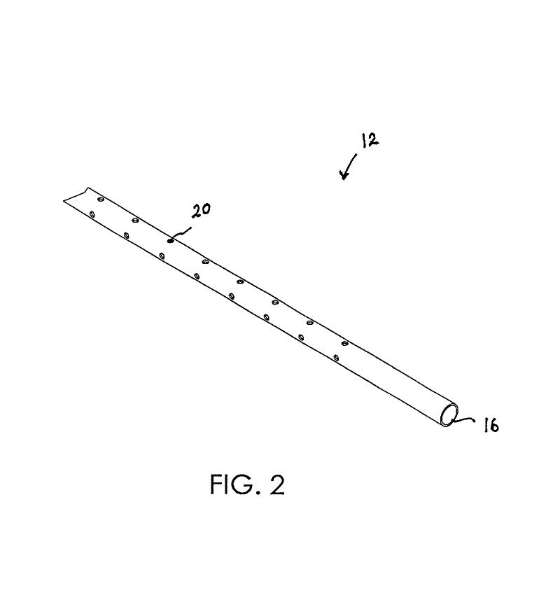

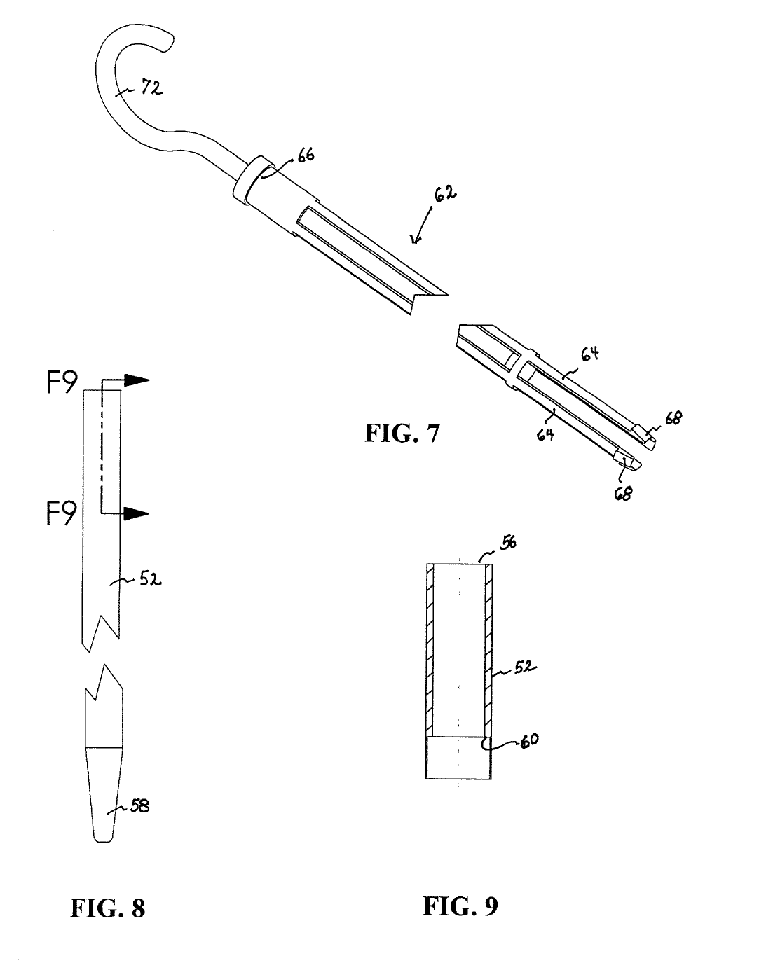

A scent dispensing device 10 according to one embodiment of the present invention will now be described in detail with reference to FIGS. 1 through 5 of the accompanying drawings. The scent dispensing device 10 includes a cylindrical first sleeve 12, also referred to as the outer sleeve, having a closed first end 14 and defining an open second end 16 (FIG. 2). The first sleeve 12 is constructed of a durable material such as carbon, steel, or fiberglass. An arrow tip 18 is fixedly attached to the closed first end 14 so that the first sleeve 12 may be inserted into a ground surface (FIG. 1), as to be described in further detail below. The first sleeve 12 defines a plurality of apertures 20 spaced apart longitudinally between the first 14 and second 16 ends. Preferably, the apertures 20 are configured in multiple linear rows parallel to one another although a single linear configuration of apertures between the ends would also work.

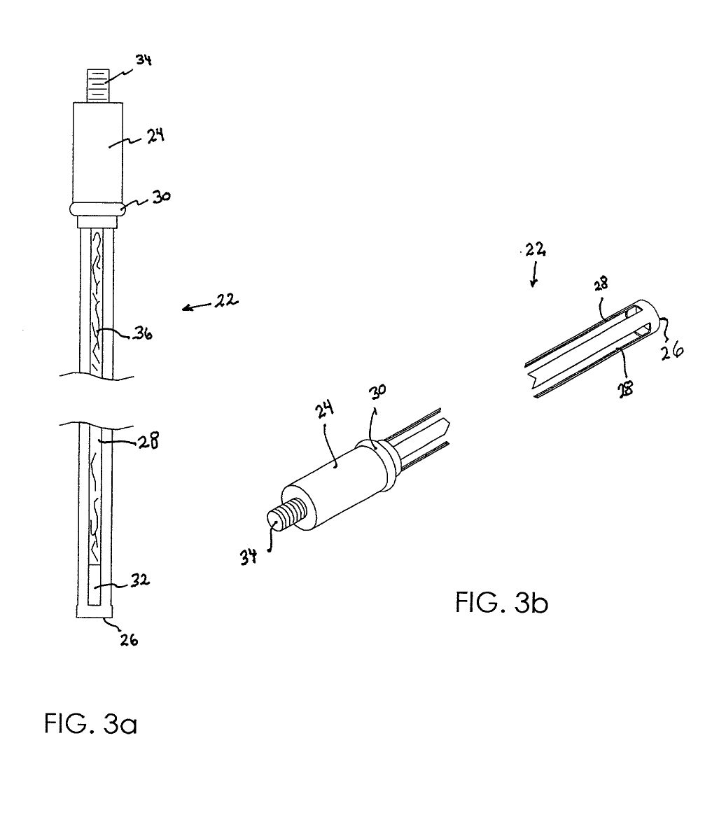

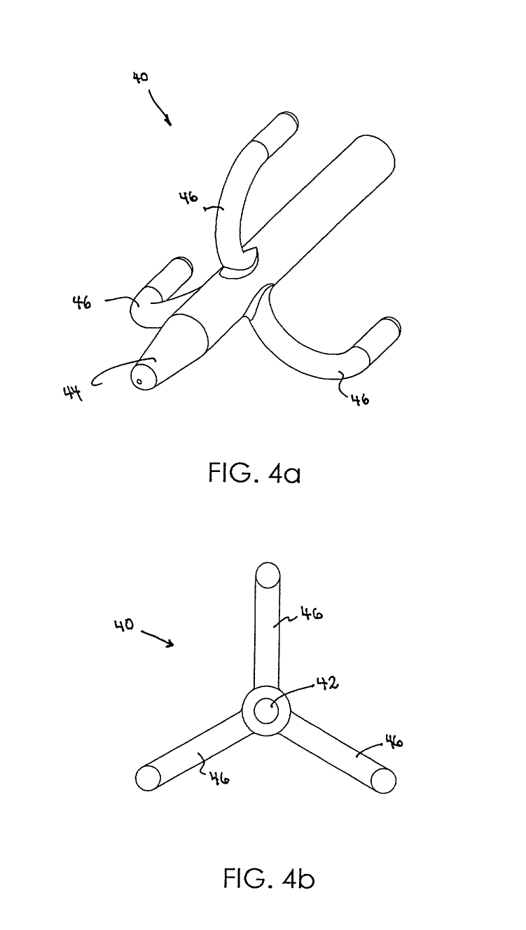

The scent dispensing device 10 further includes a second sleeve 22, also referred to as the inner sleeve, having a cylindrical configuration. The second sleeve 22 has a configuration complementary to that of the first sleeve 12 but includes a diameter smaller than a diameter of the first sleeve 12 so as to be slidable therein. As best shown in FIGS. 3a and 3b, the second sleeve 22 includes a handle member 24 at a first end and defines an oppositely disposed open second end 26. The handle member 24 enables a user to rotate the second sleeve 22 within the first sleeve 12. The second sleeve 22 defines a pair of elongate slots 28 extending longitudinally between the handle member 24 and the open second end 26. A stopper 30 is fixedly attached to the handle member 24 adjacent the slots, the rubber stopper 30 having a diameter larger than a diameter of the first sleeve 12 so as to preclude the handle member 24 from being received therein. The stopper 30 is preferably constructed of rubber so as to form a seal when the second sleeve 22 is completely slidably inserted into the first sleeve 12, as to be described more fully later.

The second sleeve 22 includes a plug 32 configured for selective insertion into the open second end 26 thereof. This plug 32 is also made of rubber and acts as a seal when inserted. With the plug removed, an absorbent material 36 such as a length of cotton may be inserted into the second sleeve 22. This absorbent material 36 may be soaked with a selected hunting scent for retaining the scent within the device 10.

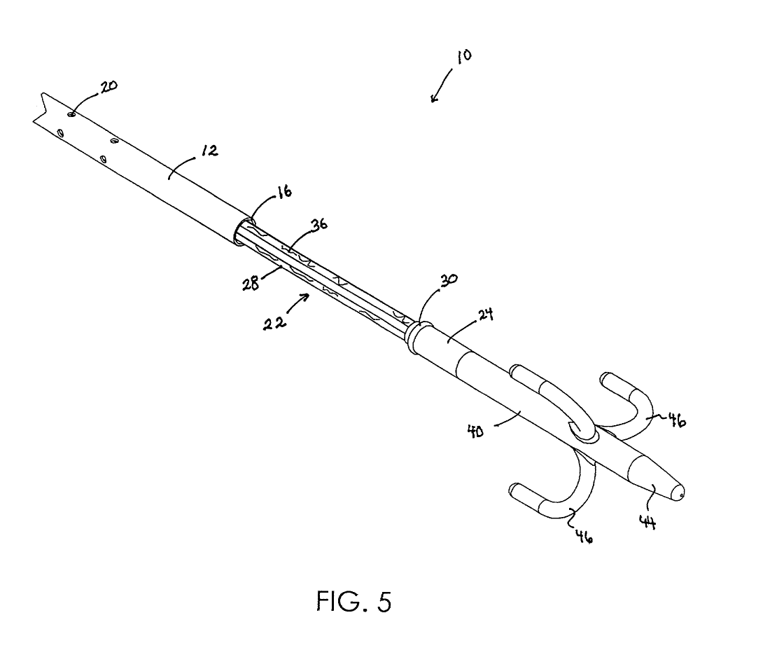

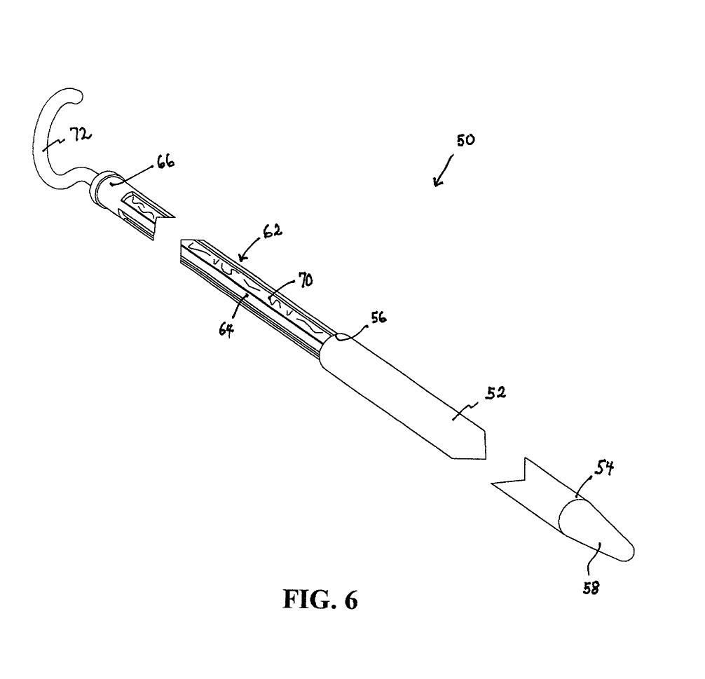

A threaded fastener 34 is fixedly attached to a free end of the handle member 24 and extends longitudinally therefrom (FIG. 3b). The scent dispensing device 10 includes a hook member 40 preferably having a plurality of generally U-shaped hanging prongs 46 extending in different directions although only a single prong is essential (FIG. 4a). The hook member 40 defines a threaded bore 42 for selectively coupling the hook member 40 to the threaded fastener 34 of the second sleeve 22 (FIG. 4b). Therefore, the dispensing device 10 may be hung from a tree branch or other similar structure. Opposite the bore 42, an arrow tip 44 is fixedly attached to a free end of the hook member 40 such that the scent dispensing device 10 may be inserted hook member first into a ground surface.

In use, the second sleeve 22 may be slidably removed from the first sleeve 12 by pulling on the handle member 24 or hook member 40. With the second sleeve 22 partially or completely removed and the plug 32 removed, liquid scent may be applied to a strip of cotton or other absorbent material 36 and inserted into the second sleeve 22. It should be appreciated that the absorbent material is removable and replaceable. The second sleeve 22 may then be slidably inserted into the first sleeve 12 through the open second end 16 thereof, the stopper 30 providing a friction fit. The stopper 30 and plug 32 effectively seal the scent within the first sleeve 12. Using the handle member 24, a user may rotate the second sleeve 22 between a first configuration in which the slots 28 and apertures 20 are not aligned and a second configuration in which the slots 28 and apertures 20 are aligned. In the first configuration, the scent odor is retained within the first sleeve 12 and is not dispensed whereas the scent odor is communicated into the ambient air when the slot 28 and apertures 20 are aligned appropriately. Either during use or storage, the scent dispensing device 10 may be hung from a tree using the hook member 40 or inserted into the ground using either arrow tip 18, 44.

An alternative embodiment 50 of the dispensing device is shown in FIGS. 6-9. This embodiment 60 includes a cylindrical outer sleeve 52 having a closed end 54 and defining an open end 56. An arrow tip 58 is fixedly attached to the closed end 54 and a stop 60 is formed internally adjacent the open end 56 (FIG. 9). This embodiment 50 further includes an inner sleeve 62 having a pair of longitudinally extending structural members 64 for positioning and retaining an absorbent material 70 therein (FIGS. 6 and 7). The structural members 64 cooperate to form an open end of the inner sleeve 62 (FIG. 7), each end having a flange 68 for mating with the stop 60 of the outer sleeve 52 . A hook 72 is fixedly attached to an end 66 of the inner sleeve 62 opposite the open end defined by the structural members 64. In use, the inner sleeve 62 is slidable between a closed configuration in which the absorbent material 70 which may be soaked with animal scent is held within the outer sleeve 52 and a second configuration in which the inner sleeve 62 is slidably extended such that the device 60 may be hung from a tree by the hook 72. The flanges 68 bear against the stop 60 when in the second configuration such that the inner sleeve 62 is not completely released from the outer sleeve 52, at least not without the application of significant pulling force.

It is understood that while certain forms of this invention have been illustrated and described, it is not limited thereto except insofar as such limitations are included in the following claims and allowable functional equivalents thereof.

Having thus described the invention, what is claimed as new and desired to be secured by Letters Patent is as follows:

1. A scent dispensing device, comprising:
a first sleeve having a closed first end and defining an open second end, said first sleeve defining a plurality of apertures spaced apart longitudinally between said first and second ends in a linear configuration;
a second sleeve slidably received in said first sleeve through said open second end, said second sleeve defining at least one slot extending longitudinally therealong;
an absorbent material selectively positioned within said second sleeve for retaining a selected scent; and
said second sleeve being rotatable in said first sleeve between a first configuration in which said slot is not aligned with said apertures such that said scent is not communicated therethrough and a second configuration in which said slot is aligned with said apertures such that said scent is dispensed therethrough.

2. The scent dispensing device as in claim 1 wherein said second sleeve further comprises:
a handle member fixedly attached to a first end of said second sleeve, said handle member adapted to be selectively rotated by a user whereby to rotate said second sleeve between said first and second configurations within said first sleeve; and
a stopper connected to said handle member for precluding said handle member from being received into said first sleeve and for sealing said selected scent within said second sleeve.

3. The scent dispensing device as in claim 2 wherein said second sleeve defines an open second end opposite said first end and includes a plug removably positioned in said open second end thereof for selectably sealing said selected scent within said second sleeve.

4. The scent dispensing device as in claim 1 further comprising:
means for sealing a first end of said second sleeve for precluding leakage of said selected scent from said first end of said second sleeve; and
means for removably sealing a second end of said second sleeve such that said absorbent material and said selected scent may be selectively deposited into or removed from said second sleeve.

5. The scent dispensing device as in claim 1 wherein said first end of said first sleeve includes an arrow tip adapted to penetrate a ground surface.

6. The scent dispensing device as in claim 1 further comprising a hook member coupled to said second sleeve, said hook member having at least one generally U-shaped hanging prong.

7. The scent dispensing device as in claim 6 wherein said hook member includes an arrow tip adapted to penetrate a ground surface.

8. The scent dispensing device as in claim 1 wherein said first shaft is constructed of steel.

9. A scent dispensing device, comprising:
a first sleeve having a closed first end and defining an open second end, said first sleeve defining a plurality of apertures spaced apart longitudinally between said first and second ends in a linear configuration;
wherein said closed first end includes a first arrow tip adapted to penetrate a ground surface;
a second sleeve slidably received in said first sleeve through said open second end, said second sleeve defining at least one slot extending longitudinally therealong;
an absorbent material selectively positioned within said second sleeve for retaining a selected scent; and
said second sleeve being rotatable in said first sleeve between a first configuration in which said slot is not aligned with said apertures such that said scent is not communicated therethrough and a second configuration in which said slot is aligned with said apertures such that said scent is dispensed therethrough;
a hook member coupled to said second sleeve and having at least one generally U-shaped hanging prong; and
a second arrow tip depending from a free end of said hook member and adapted to penetrate a ground surface.

10. The scent dispensing device as in claim 9 wherein said second sleeve further comprises:
a handle member fixedly attached to a first end of said second sleeve, said handle member adapted to be selectively rotated by a user whereby to correspondingly rotate said second sleeve between said first and second configurations within said first sleeve; and
a stopper connected to said handle member for precluding said handle member from being received into said first sleeve through said open second end thereof and for sealing said selected scent within said second sleeve.

11. The scent dispensing device as in claim 10 wherein said second sleeve defines an open second end opposite said first end and includes a plug removably positioned in said open second end thereof for selectably sealing said selected scent within said second sleeve.

12. The scent dispensing device as in claim 9 further comprising:
means for sealing a first end of said second sleeve for precluding leakage of said selected scent from said second sleeve; and
removable means for sealing a second end of said second sleeve such that said absorbent material and said selected scent may be selectively deposited into or removed from said second sleeve.

13. The scent dispensing device as in claim 9 wherein said first sleeve is constructed of a carbon material.

14. A scent dispensing device, comprising:
a first sleeve having a closed first end and defining an open second end, said first sleeve defining a plurality of apertures spaced apart longitudinally between said first and second ends in a linear configuration;
wherein said closed first end includes a first arrow tip adapted to penetrate a ground surface;
a second sleeve slidably received in said first sleeve through said open second end, said second sleeve defining at least one slot extending longitudinally therealong;
an absorbent material selectively positioned within said second sleeve for retaining a selected scent;
said second sleeve being rotatable in said first sleeve between a first configuration in which said slot is not aligned with said apertures such that said scent is not communicated therethrough and a second configuration in which said slot is aligned with said apertures such that said scent is dispensed therethrough;
a handle member attached to a first end of said second sleeve, said handle member adapted to be selectively rotated by a user so as to correspondingly rotate said second sleeve between said first and second configurations within said first sleeve;
removable means for sealing a second end of said second sleeve such that said absorbent material and said selected scent may be selectively deposited into or removed from said second sleeve;
a flange extending from said handle member, said flange having a plurality of threads;
a hook member defining a bore threadably coupled to said flange and having at least one generally U-shaped hanging prong; and
a second arrow tip depending from a free end of said hook member and adapted to penetrate the ground surface.

15. A scent dispensing device as in claim 14 further comprising a stopper fixedly attached to said first end of said second sleeve adjacent said handle member, said stopper adapted to preclude said handle member from being received into said first sleeve and to seal said deposited scent within said second sleeve.

16. The scent dispensing device as in claim 14 wherein said first sleeve is constructed of a fiberglass material.

17. A scent dispensing device, comprising:
a first sleeve having a closed first end and defining an open second end;
a second sleeve slidably received in said first sleeve through said open second end of said first sleeve, said second sleeve having a closed first end and having a framework depending longitudinally therefrom;
an absorbent material selectively positioned within said framework of said second sleeve for retaining a selected scent; and
said second sleeve being slidable in said first sleeve between a first configuration in which said framework is completely retracted within said first sleeve and a second configuration in which said second sleeve is substantially extended from said open second end of said first sleeve such that said scent is released into an ambient air.

18. The scent dispensing device as in claim 17 wherein:
said first sleeve includes a stop portion adjacent said open second end thereof having a diameter that is smaller than a diameter of a main portion of said first sleeve; and
said framework includes a pair of flanges at a free end opposite said closed first end of said second sleeve, said pair of flanges adapted to bear against said stop portion of said first sleeve at said second configuration, whereby to preclude complete removal of said second sleeve from said first sleeve.

19. The scent dispensing device as in claim 17 further comprising a hook member coupled to said closed first end of said second sleeve.
